(19) United States
(12) Patent Application Publication
(10) Pub. No.: US 2003/0106076 A1
(43) Pub. Date: Jun. 5, 2003

(54) Methods for identifying genes expressed in selected lineages, and a novel genes identified using the methods

(75) Inventors: William Stanford, Toronto (CA); Georgina Caruana, Toronto (CA); Michihiro Hidaka, Kumamoto (JP); Alan Bernstein, Toronto (CA)

Correspondence Address: MERCHANT & GOULD PC, P.O. BOX 2903, MINNEAPOLIS, MN, 55402-0903, US

(73) Assignee: Mount Sinai Hospital Corporation, Toronto, ON (CA)

(21) Appl. No.: 10/194,746

(22) Filed: Jul. 12, 2002

Related U.S. Application Data
(63) Continuation of application No. 09/462,772, filed on Apr. 10, 2000, now abandoned.
Application No. 09/462,772 is a 371 of international application No. PCT/CA98/00667, filed on Jul. 10, 1998.
(60) Provisional application No. 60/052,293, filed on Jul. 11, 1997.

(51) Int. Cl. A01K 67/00; C12Q 1/68; C07H 21/04; C12P 21/02; C12N 5/06

(52) U.S. Cl. 800/8; 435/6; 435/455; 435/69.1; 435/320.1; 435/325; 536/23.2

(57) ABSTRACT

The invention relates to vectors, compositions, and methods for identifying genes primarily expressed in selected lineages. The invention also relates to novel genes primarily expressed in selected lineages, proteins encoded by the novel genes and truncations, analogs, homologs, and isoforms of the proteins and uses of the proteins and genes.

### FIELD OF THE INVENTION

The invention relates to vectors, compositions, and methods, for identifying genes primarily expressed in selected lineages. The invention also relates to novel genes primarily expressed in selected lineages, proteins encoded by the novel genes and truncations, analogs, homologs, and isoforms of the proteins; and, uses of the proteins and genes.

### BACKGROUND OF THE INVENTION

Gene trapping strategies have been used to identify eukaryotic genes displaying novel and familiar patterns of expression during embryogenesis (D. P. Hill and W. Wurst, Methods in Enzymology, 225: 664, 1993). The techniques use vectors which are randomly integrated into genes. The vectors typically contain a reporter gene which facilitates the identification and isolation of the vectors once they are inserted into a gene. Gene trap vectors also typically contain sequences associated with eukaryotic structural genes such as splice-acceptor sites which occur at the 5′ end of all exons. Vectors containing a splice-acceptor site integrate into introns and generate a fusion transcript containing a target endogenous gene and the reporter gene (see references 5, 10, 11 in D. P. Hill and W. Wurst, Supra). The expression of the reporter gene is under the regulatory control of the endogenous gene and its expression mimics the expression pattern of the target gene (see reference 12 in D. P. Hill and W. Wurst, Supra). The insertion of the gene trap vector can also create a mutation and disrupt the function of the target gene (see references 10 and 12 in D. P. Hill and W. Wurst, 20 Supra). The part of the target gene in the fusion transcript may also be cloned from the fusion transcript, or from genomic DNA upstream of the insertion site.

Embryonic stem (ES) cell technology offers an efficient way of introducing gene trap vectors into the mouse genome and thereby identify and mutate genes expressed during mouse development. ES cells isolated from the mouse inner cell mass remain pluripotent after genetic manipulation and in vitro culture, and they contribute to all tissues of the mouse, including the germ line (see references 7 to 9 in D. P. Hill and W. Wurst, Supra).

Different approaches have been used to identify targeted genes using ES technology. Mutations can be transmitted through the germ line and offspring can be screened for recessive mutant phenotypes. Prescreening in chimeric embryos can also be carried out, and mutations resulting in interesting patterns can be transmitted through the germ line and their phenotype studied.

Gene trapping in ES cells is a powerful technique because it simultaneously integrates gene identification and structure, expression and functional analysis into one process. Typically gene trap screens have used one of these three types of analyses as the primary determinant to select clones for further study. The first group of screens uses no pre-selection to study mutant phenotypes. Collectively, these studies have determined that nearly 40% of gene trap mutants result in recessive embryonic lethality [Friedrich G, Genes & Dev. 5:1513, 1991; Skarnes W C, INSERT1992; von Melchner H, Genes & Dev. 6:919, 1992; DeGregori J, Genes & Dev. 8:265, 1994). Several sequence-based screening strategies have been developed to either rapidly isolate 5′RACE sequences (Holzschu D, Transgenic Res. 6:97, 1997; Chowdhury K, Nucleic Acid Res. 25:1531, 1997; and Townley D J, Genome Res. 7:293, 1997), isolate 3′RACE sequences (Yoshida M. et al, Trans. Res. 4:277, 1995; and Zambrowicz B P et al, Nature 392:608, 1998), or clone proviral integraton sites by plasmid rescue (Hicks G G et al Nature Genet. 16:338, 1997). In addition Skarnes and colleagues modified the GT1.8geo vector to specifically trap genes which encode secreted or transmembrane proteins (Proc. Natl. Acad. Sci. USA 92:6592, 1995). Several groups have performed screens based upon regulated expression. Each of these screens analyzed clones which contained integrations into genes which were transcriptionally active in ES cells. The expression of the fusion transcripts were either analyzed by in vivo expression (Wurst W, Genetics 139:889, 1995), regulation by exogenous factors (Sam M et al, Dev. Dyn; Forrester L et al, Proc. Natl. Acad. USA 93:1677, 1996; Sam M et al, Mann. Genome 7:741, 1996), or by in vitro differentiation (Scherer C A et al, Cell Growth & Diff. 7:1393, 1996; Shirai M et al, Zool. Sci. 13:277, 1996; and Baker R K et al, Dev. Biol 185:201, 1997).

### SUMMARY OF THE INVENTION

The present inventors have developed a gene trap strategy to identify, mutate, and characterize large numbers of genes on the basis of their cell-lineage specific expression. This expression trapping method complements and extends previous expression-based gene trap screens by specifically identifying integrations into genes preferentially expressed in selected cell lineages. The approach simultaneously provides expression, sequence, and phenotypic information. The method can be used to carry out large scale, genome-wide scans for genes of interest. Integrations with identifiable expression patterns in vitro can be catalogued to generate a biological resource of gene-trap insertions, based upon expression pattern, cDNA sequences, and mutant phenotypes. The method permits identification of specific messages present in low levels that could not have been found using conventional techniques.

Therefore, broadly stated the present invention relates to a method of identifying a target nucleic acid molecule primarily expressed in selected lineages comprising:

(a) integrating into a site in the genome of a host cell a gene trap vector containing a reporter gene, to form transfected cells;

(b) growing the transfected cells in vitro under conditions whereby the transfected cells differentiate into embryoid bodies attached to a carrier and identifying embryoid bodies expressing the reporter gene in cells of a selected lineage, or

(c) growing the transfected cells in vitro under conditions whereby the transfected cells differentiate into cells of a selected lineage, and identifying cells of the selected lineage expressing the reporter gene;

wherein the target nucleic acid molecule comprises sequences upstream or downstream of the site of integration of the reporter gene in the cells of the selected lineage.

The method may further comprise isolating nucleic acid molecules from the transfected cells, or descendents thereof expressing the reporter gene wherein the nucleic acid molecules comprise the reporter gene and a part of the target nucleic acid molecule, or the nucleic acid molecules comprise genomic DNA upstream or downstream of the site of insertion of the gene trap vector.

Transfected cells or descendents thereof expressing the reporter gene may be introduced into embryos to form chimeric embryos. Therefore, the present invention contemplates a chimeric embryo having integrated into its genome a gene trap vector at a site of a target nucleic acid molecule primarily expressed in cells of selected lineages. Germline transmission may be achieved by mating chimeric embryos allowed to mature to term, or mating foster recipient females having the chimeric embryos. Therefore, the invention also contemplates a transgenic non-human animal all of whose somatic cells and germ cells contain a gene trap vector at a site of a target gene primarily expressed in cells of selected lineages.

The present inventors using the novel strategy described herein have identified novel clones expressed primarily in hematopoietic, endothelial, stromal, and/or myocyte lineages designated 17G2, K18F2, K20D4, K18F2, K20D4, B2D2, GC10E10, GC11C7, and GC11E10. The invention therefore relates to novel nucleic acid molecules isolated from these clones.

The nucleic acid molecules of the invention permit identification of untranslated nucleic acid sequences or regulatory sequences which specifically promote expression of proteins operatively linked to the promoter regions. Identification and use of such promoter sequences are particularly desirable in instances, such as gene transfer or gene therapy, which can specifically require heterologous gene expression in a limited (e.g. hematopoietic or vascular) environment. The invention therefore contemplates a nucleic acid encoding a regulatory sequence of a nucleic acid molecule of the invention, such as a promoter sequence.

The nucleic acid molecules of the invention may be inserted into an appropriate vector, and the vector may contain the necessary elements for the transcription and translation of the inserted coding sequence. Accordingly, vectors may be constructed which comprise a nucleic acid molecule of the invention and optionally one or more transcription and translation elements linked to the nucleic acid molecule.

Vectors are contemplated within the scope of the invention which comprise regulatory sequences of the invention, as well as chimeric gene constructs wherein a regulatory sequence of the invention is operably linked to a nucleic acid sequence encoding a heterologous protein, and a transcription termination signal.

A vector of the invention can be used to prepare transformed host cells expressing the proteins encoded by the nucleic acids of the invention, or a heterologous protein. Therefore, the invention further provides host cells containing a vector of the invention. The invention also contemplates transgenic non-human mammals whose germ cells and somatic cells contain a vector comprising a nucleic acid molecule of the invention or a fragment thereof, in particular one which encodes an analog or a truncation of a protein of the invention.

The invention further provides a method for preparing novel proteins encoded by the nucleic acids of the invention utilizing the purified and isolated nucleic acid molecules of the invention. In an embodiment a method for preparing a protein is provided comprising (a) transferring a vector of the invention into a host cell; (b) selecting transformed host cells from untransformed host cells; (c) culturing a selected transformed host cell under conditions which allow expression of the protein; and (d) isolating the protein. A protein of the invention may be obtained as an isolate from natural cell sources, but they are preferably obtained by recombinant procedures.

The invention further broadly contemplates an isolated protein comprising the amino acid sequence of SEQ. ID. NO.2, SEQ. ID. NO 5., or SEQ. ID. NO. 7. The invention includes a truncation of a protein of the invention, an analog, an allelic or species variation thereof, or a homolog of a protein of the invention, or a truncation thereof. ( The term “proteins of the invention” used herein includes truncations, analogs, allelic or species variations, and homologs).

The proteins of the invention may be conjugated with other molecules, such as proteins, to prepare fusion proteins or chimeric proteins. This may be accomplished, for example, by the synthesis of N-terminal or C-terminal fusion proteins.

The invention further contemplates antibodies having specificity against an epitope of a protein of the invention. Antibodies may be labelled with a detectable substance and used to detect proteins of the invention in tissues and cells.

The invention also permits the construction of nucleotide probes which are unique to the nucleic acid molecules of the invention. Therefore, the invention also relates to a probe comprising a sequence derived from a nucleic acid of the invention or encoding a protein of the invention. The probe may be labelled, for example, with a detectable substance and it may be used to select from a mixture of nucleotide sequences a nucleic acid sequence of the invention, or a nucleic acid sequence encoding a protein of the invention.

The invention still further provides a method for identifying a substance which binds to a protein of the invention comprising reacting a protein with at least one substance which potentially can bind with the protein, under conditions which permit the formation of complexes between the substance and protein and assaying for complexes, for free substance, for non-complexed protein, or for activated protein.

Still further the invention provides a method for evaluating a compound for its ability to modulate the biological activity of a protein of the invention. For example a substance which inhibits or enhances the interaction of the protein and a substance which binds to the protein may be evaluated. In an embodiment, the method comprises providing a known concentration of a protein, with a substance which binds to the protein and a test compound under conditions which permit the formation of complexes between the substance and protein, and assaying for complexes, for free substance, for non-complexed protein, or for activated protein.

Compounds which modulate the biological activity of a nucleic acid or protein of the invention may also be identified using the methods of the invention by comparing the pattern and level of expression of nucleic acid or protein of the invention in tissues and cells, in the presence, and in the absence of the compounds.

The substances and compounds identified using the methods of the invention may be used to modulate a nucleic acid or protein of the invention, and they may be used in the treatment of conditions requiring modulation of for example hematopoiesis, myocardium, the sensory nervous system, or cardiac or neural vasculature. Accordingly, the substances and compounds may be formulated into compositions for administration to individuals suffering from one of these conditions. Therefore, the present invention also relates to a composition comprising one or more of a protein of the invention, or a substance or compound identified using the methods of the invention, and a pharmaceutically acceptable carrier, excipient or diluent. A method for treating or preventing a condition requiring modulation of hematopoiesis, the sensory nervous system, or vasculature is also provided comprising administering to a patient in need thereof, a protein of the invention or a composition of the invention.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DESCRIPTION OF THE DRAWINGS

The invention will be better understood with reference to the drawings in which:

FIG. 1, panels A to I are photographs showing K17G2-lacZ expression in vitro and in vivo;

FIG. 2, panels A to I are photographs showing GC11E10-lacZ expression;

FIG. 3, panels A to F, are photographs showing Mena-lacZ (K18E2) expression.

### DETAILED DESCRIPTION OF THE INVENTION

1. Expression Trapping Method

As hereinbefore mentioned, the present invention provides a method for detecting a target nucleic acid molecule primarily expressed in selected lineages. In an embodiment of the invention the target nucleic acid molecule is primarily expressed in hematopoietic or endothelial cells.

The term “hematopoiesis” used herein refers to the proliferation, differentiation, and migration of hematopoietic cells in embryos and adults. “Hematopoietic cells” refers to cells of the hematopoietic system including pluripotential stem cells which are capable of self-replication and of differentiation to committed progenitor cells; progenitor cells; myeloid and lymphoid stem cells; and neutrophils, macrophages, erythroid cells, mast cells, megakaryocytes, blast cells, lymphocytes, and monocytes. “Endothelial cells” refers to a type of squamous epithelium cells that lines the interiors of cavities, spaces, and blood vessels.

The method of the invention involves integrating into the genomes of host cells a gene trap vector containing a reporter gene, to form transfected cells. The gene trap vector used in the method of the invention comprises a reporter gene which allows for differentiation of cells having a gene trap vector integrated into a target nucleic acid molecule primarily expressed in selected lineages (e.g. hematopoietic or endothelial cells). Reporter genes which are particularly useful in the method of the invention are genes encoding β-galactosidase (e.g. lac Z), chloramphenicol, acetyltransferase, or firefly luciferase. Transcription of the reporter gene is monitored by changes in the concentration of the protein encoded by the reporter gene such as β-galactosidase, chloramphenicol, acetyltransferase, green fluorescence protein (GFP), or firefly luciferase. Transfected cells or descendents thereof showing reporter gene activity are identified using conventional methods. For example, if the reporter gene encodes β-galactosidase, activity can be analyzed by staining with 5-bromo-4-chloro 3-indolyl galactoside as described in Proc. Natl Acad, Sci. USA 84: 156, 1987.

The gene trap vector may also include a gene encoding a selectable marker which conveys a second property on transformed cells and permits the selection and/or identification of cells having the vector integrated into their genome. Examples of such genes are genes which encode proteins conferring antibiotic resistance, or the ability to grow on a defined medium. For example, a gene encoding neomycin (neo) phosphotransferase activity and conferring neomycin resistance may be included in the gene trap vector.

The differentiation and selection of cells using a reporter gene and selectable marker gene may be achieved using a single element. For example, a β-geo construct which has sequences conferring both β-galactosidase and neomycin (neo) phosphotransferase activities may be incorporated into the gene trap vector.

The gene trap vector may include regulatory sequences such as promoter sequences which control the expression of one or both of the reporter gene and selectable marker gene. The reporter gene or selectable marker gene may not be under the control of an autonomous promoter, and they may only be expressed if the gene trap vector is integrated into an actively expressed gene.

The gene trap vector may include sequences associated with eukaryotic structural genes which facilitate the insertion of the vector into a eukaryotic gene. For example, the gene trap vector may include sequences associated with elimination of intron sequences from mRNA such as splicer-acceptor sequences (e.g. using an En entron), and polyadenylation signal sequences.

The gene trap vector may also include sequences which facilitate isolation and sequencing of the target gene. For example, the gene trap vector may contain loxp sequences before and after the lacZ sequence. The loxp sequences are cleaved by cre recombinase allowing removal of the lacZ sequence.

Preferred gene trap vectors for use in the method of the invention are PT1 which contains an En-2 intron sequence including a splice-acceptor site in front of the bacterial lacZ gene and a neomycin gene driven by the PGK-I promoter; PT1/ATG which is the same as PT1 with the exception that it includes a translational start signal (ATG) in the lacZ gene (Hill D P and Wurst W, Methods in Enzymology 225:664, 1993); and GT1.8geo which contains the En-2 splice acceptor site immediately upstream of a lacZ-neo vector thereby allowing neomycin resistance at a lower level of endogenous gene expression than the SAβgeo vector (Skarnes W C et al., Proc. Natl. Acad. Sci. USA 92:652-6596, 1995).

The gene trap vector may be introduced into host cells by conventional methods such as transfection, lipofection, precipitation, infection, electroporation, nucroinjection etc. Methods for transfecting, etc. host cells are well known in the art (see Sambrook et al. Molecular Cloning A Laboratory Manual, 2nd edition, Cold Spring Harbor Laboratory Press, 1989, all of which is incorporated herein by reference).

Suitable host cells for use in the method of the invention include a wide variety of host cells, including stem cells, and pluripotent cells such as zygotes, embryos, and ES cells, preferably ES cells. The gene trap vector stably integrates into the genome of the host cells. Generally, the vector integrates randomly into the genome of the host cells and in some cells it will integrate into endogenous genes which are primarily expressed in hematopoietic or endothelial cells.

The transfected host cells containing the gene trap vector may be grown in vitro under conditions whereby the transfected cells differentiate into embryoid bodies. Methods for producing EB culture systems are known to the skilled artisan. See for example, Bautch VL. Et al, Dev. Dyn. 205:1-12, 1996. Preferably the embryoid bodies are grown attached to a carrier or support so that the endoderm layer is beneath the blood islands. The carrier or support may be made of nitrocellulose, glass, polyacrylamide, gabbros, o magnetite. The support or carrier material may have any possible configuration including spherical (e.g. bead), cylindrical (e.g. inside surface of a test tube or well, or the external surface of a rod), or flat (e.g. sheet, test strip).

The transfected host cells containing the gene trap vector may be grown in vitro under conditions selected so that the transfected cells differentiate into cells of a selected lineage, and the reporter gene is expressed in the transfected cells. For example, host cells which are embryonic stem cells may be cultured with a cell line which induces differentiation of the embryonic stem cells into hematopoietic cells such as the OP9 stromal cell line described by Nakano et al., (Science 265:1098, 1994). The methods of the invention can also be adapted to identify target nucleic acid molecules primarily expressed in particular cell types by adding one or more exogenous factors (e.g. cytokines) which induce the differentiation of specific cell types. For example, to identify and isolate nucleic acid molecules associated with differentiation of macrophages-granulocytes, transfected host cells containing a gene trap vector may be grown on OP9 cell layers in the presence of granulocyte-macrophage colony-stimulating factor.

In a preferred embodiment of the invention embryonic stem cells transfected with a gene trap vector containing a β-galactosidase gene and a gene conferring antibiotic resistance are seeded onto confluent OP9 cell layers on well plates at a concentration of 10³ to 10⁵, preferably 10⁴ cells per well. The induced cells are trypsinized between day 5 and day 8, preferably day 5. β-galactosidase activity is observed in the induced cells between about day 5 and day 12.

Nucleic acid molecules containing the reporter gene and a part of the target gene, or containing genomic DNA upstream or downstream of the site of integration of the gene trap vector, may be isolated and cloned using standard methods from the transfected cells, or descendents thereof showing reporter gene activity. Cloned nucleic acid molecules may be sequenced and the predicted amino acid sequence of the encoded protein can be determined using standard sequencing techniques, such as dideoxynucleotide chain termination, or Maxam-Gilbert chemical sequencing. The initiation codon and untranslated sequences of the protein may be determined using currently available computer software designed for the purpose, such as PC/Gene (IntelliGenetics Inc., Calif.). The intron-exon structure and transcription regulatory sequences of a gene can be identified using conventional techniques.

Transfected cells or descendents thereof expressing the reporter gene may be used to generate chimeric embryos. For example, clones showing reporter gene activity can be aggregated with diploid embryos (e.g. Nagy, A and Rossant J. In A. L. J. (ed): Gene Targeting: A practical Approach. Oxford, IRL, 1993, p. 147-178), and allowed to mature to term. Chimeric mice can be mated (e.g. to CD-1 mice) to provide animal lines having the mutation transmitted through the germline. Such a transgenic animal may be used to study the phenotype produced by the interruption of an endogenous gene by the gene trap vector, and to identify substances that reverse or enhance such a mutation.

2. Nucleic Acid Molecules and Proteins Identified Using the Methods of the Invention

2.1 Nucleic Acid Molecules

As hereinbefore mentioned, the invention provides an isolated nucleic acid molecule having a sequence encoding a novel protein of the invention. The term “isolated” refers to a nucleic acid substantially free of cellular material or culture medium when produced by recombinant DNA techniques, or chemical reactants, or other chemicals when chemically synthesized. An “isolated” nucleic acid is also free of sequences which naturally flank the nucleic acid (i.e., sequences located at the 5′ and 3′ ends of the nucleic acid molecule) from which the nucleic acid is derived. The term “nucleic acid” is intended to include DNA and RNA and can be either double stranded or single stranded.

The invention specifically contemplates an isolated nucleic acid molecule which comprises:

(i) a nucleic acid sequence encoding a protein having substantial sequence identity preferably at least 75% sequence identity, with the amino acid sequence of SEQ. ID. NO.2, SEQ. ID. NO 5., or SEQ. ID. NO. 7;

(ii) nucleic acid sequences complementary to (i);

(iii) a degenerate form of a nucleic acid sequence of (i);

(iv) a nucleic acid sequence comprising at least 18 nucleotides and capable of hybridizing to a nucleic acid sequence in (i), (ii), or (iii);

(v) a nucleic acid sequence encoding a truncation, an analog, an allelic or species variation of a protein comprising the amino acid sequence shown SEQ. ID. NO.2, SEQ. ID. NO 5., or SEQ. ID. NO. 7; or

(vi) a fragment, or allelic or species variation of (i), (ii) or (iii).

In an embodiment of the invention a nucleic acid molecule is provided comprising:

(i) a nucleic acid sequence comprising the sequence of SEQ. ID. NO.1, SEQ. ID. NO 3., SEQ. ID. NO. 4, SEQ. ID. NO. 6, SEQ. ID. NO. 8, SEQ. ID. NO. 9, or SEQ. ID. NO. 10, wherein T can also be U;

(ii) nucleic acid sequences complementary to (i), preferably complementary to the full nucleic acid sequence of SEQ. ID. NO.1, SEQ. ID. NO 3., SEQ. ID. NO. 4, SEQ. ID. NO. 6, SEQ. ID. NO. 8, SEQ. ID. NO. 9, or SEQ. ID. NO. 10;

(iii) a nucleic acid capable of hybridizing to a nucleic acid of (i) and having at least 18 nucleotides; or

(iv) a nucleic acid molecule differing from any of the nucleic acids of (i) to (iii) in codon sequences due to the degeneracy of the genetic code.

In accordance with specific embodiments of the invention the following nucleic acid molecules or genes are provided

(a) A novel nucleic acid molecule designated 17G2 which is primarily expressed in vivo in hematopoietic cells, myocardium, in the cardiac and neural vasculature, and in the sensory nervous system, including the trigeminal ganglia, dorsal root ganglia, and optic nerve. The nucleic acid molecule comprises the sequence of SEQ.ID. No. 1.

(b) A novel nucleic acid molecule designated K18F2 which is primarily expressed in vitro by muscle cells in attached embryoid bodies, and some mesodermal cells in OP9 induction cultures, and primarily expressed in vivo in both tetraploid and diploid chimeric embryos exclusively in cardiac myocytes. The nucleic acid molecule comprises the sequence of SEQ.ID. No. 3.

(c) A novel nucleic acid molecule designated K20D4 which is expressed in vitro exclusively in vascular endothelial cells in attached embryoid bodies, and some mesodermal cells in OP9 induction. The nucleic acid molecule comprises the sequence of SEQ.ID. No. 4. The sequence overlaps with EST accession No. AA239055 of clone 697718 from the Barstead mouse pooled organs cDNA library.

(d) A novel nucleic acid molecule designated B2D2 which is primarily expressed in vitro in blood islands and vascular endothelial cells in attached EB cultures. However, on OP9 stroma, expression is induced in some mesodermal cells but not in hematopoietic cells. Thus, expression in the blood island may be due to endothelial cells or their precursors. The nucleic acid molecule comprises the sequence of SEQ.ID. No. 6. The sequence overlaps with EST accession No. AA209568 of clone 676502 from the Soares NML mouse liver cDNA library.

(e) A novel nucleic acid molecule designated GC10E10 which is highly expressed in vitro in undifferentiated embryonic cells. In attached embryoid bodies GC10E10 is expressed in blood islands and endothelial cells. It is expressed highly in mesodermal cells and in low levels in a population of hematopoietic cells in OP9 induction cultures. In vivo the gene is expressed in the forebrain, midbrain, sonutes, notochord, otic vesicle, limb buds, branchial arches and heart in diploid chimeras. The nucleic acid molecule comprises the sequence of SEQ.ID. No. 8. The sequence has 98% homology with the murine Dlgh1 (dlg1)

(f) A novel nucleic acid molecule designated GC11C7 which is primarily expressed in vitro in undifferentiated embryonic stem cells and in mesoderm and hematopoietic cells in the OP9 induction system. The nucleic acid molecule comprises the sequence of SEQ.ID. No. 9. The sequence overlaps that of EST accession No. AA015451, clone 442692 from the Soares mouse placenta 4NbMPI3.5 14.5 cDNA library and EST accession No. AA517189 clone 893845 from the Knowles Solter mouse embryonic stem cell cDNA library.

(g) A novel nucleic acid molecule designated GC11E10 which is highly expressed in vitro in undifferentiated embryonic stem cells and in blood islands and endothelial cells within attached embryoid bodies. It is also expressed in mesodermal cells and highly in hematopoietic cells in the OP-9 induction system. In vivo it is expressed in endothelial and blood cells within E9.5 diploid chimeras. The nucleic acid molecule comprises the sequence of SEQ.ID. No. 10.

The invention includes nucleic acid molecules having substantial sequence identity or similarity to the nucleic acid sequences of SEQ. ID. NO.1, SEQ. ID. NO 3., SEQ. ID. NO. 4, SEQ. ID. NO. 6, SEQ. ID. NO. 8, SEQ. ID. NO. 9, or SEQ. ID. NO. 10. Identity or similarity refers to sequence similarity between sequences and can be determined by comparing a position in each sequence which may be aligned for purposes of comparison. When a position in the compared sequence is occupied by the same nucleotide base or amino acid, then the molecules are matching or have identical positions shared by the sequences. Preferably, the nucleic acid sequences have substantial sequence identity for example at least 75% nucleic acid identity, more preferably 80% nucleic acid identity; and most preferably at least 90 to 95% sequence identity.

Isolated nucleic acid molecules having a sequence which differs from the nucleic acid sequence of SEQ. ID. NO.1, SEQ. ID. NO 3., SEQ. ID. NO. 4, SEQ. ID. NO. 6, SEQ. ID. NO. 8, SEQ. ID. NO. 9, or SEQ. ID. NO. 10, due to degeneracy in the genetic code are also within the scope of the invention. As one example, DNA sequence polymorphisms within the nucleotide sequence of a 17G2 protein may result in silent mutations which do not affect the amino acid sequence. Variations in one or more nucleotides may exist among individuals within a population due to natural allelic variation. Any and all such nucleic acid variations are within the scope of the invention. DNA sequence polymorphisms may also occur which lead to changes in the amino acid sequence of the protein. These amino acid polymorphisms are also within the scope of the present invention.

Another aspect of the invention provides a nucleic acid molecule which hybridizes under selective conditions, e.g. high stringency conditions, to a nucleic acid molecule of the invention. Selectivity of hybridization occurs with a certain degree of specificity rather than being random. Appropriate stringency conditions which promote DNA hybridization are known to those skilled in the art, or can be found in Current Protocols in Molecular Biology, John Wiley & Sons, N. Y. (1989), 6.3.1-6.3.6. For example, 6.0×sodium chloride/sodium citrate (SSC) at about 45° C., followed by a wash of 2.0×SSC at 50° C. may be employed. The stringency may be selected based on the conditions used in the wash step. By way of example, the salt concentration in the wash step can be selected from a high stringency of about 0.2×SSC at 50° C. In addition, the temperature in the wash step can be at high stringency conditions, at about 65° C.

It will be appreciated that the invention includes nucleic acid molecules encoding a protein of the invention including truncations, analogs and homologs of a protein of the invention as described herein. In particular, fragments of a nucleic acid molecule of the invention are contemplated that are a stretch of at least about 18 nucleotides, more typically 50 to 200 nucleotides. It will further be appreciated that variant forms of the nucleic acid molecules of the invention which arise by alternative splicing of an mRNA corresponding to a cDNA of the invention are encompassed by the invention.

An isolated nucleic acid molecule of the invention which comprises DNA can be isolated by preparing a labelled nucleic acid probe based on all or part of a nucleic acid sequence of SEQ. ID. NO.1, SEQ. ID. NO 3., SEQ. ID. NO. 4, SEQ. ID. NO. 6, SEQ. ID. NO. 8, SEQ. ID. NO. 9, or SEQ. ID. NO. 10. The labelled nucleic acid probe is used to screen an appropriate DNA library (e.g. a cDNA or genomic DNA library). For example, a cDNA library can be used to isolate a cDNA by screening the library with the labelled probe using standard techniques. Alternatively, a genomic DNA library can be similarly screened to isolate a genomic clone encompassing a gene of the invention. Nucleic acids isolated by screening of a cDNA or genomic DNA library can be sequenced by standard techniques.

An isolated nucleic acid molecule of the invention which is DNA can also be isolated by selectively amplifying a nucleic acid using polymerase chain reaction (PCR) methods and cDNA or genomic DNA. It is possible to design synthetic oligonucleotide primers from the nucleotide sequence of SEQ. ID. NO.1, SEQ. ID. NO 3., SEQ. ID. NO. 4, SEQ. ID. NO. 6, SEQ. ID. NO. 8, SEQ. ID. NO. 9, or SEQ. ID. NO. 10 for use in PCR. A nucleic acid can be amplified from cDNA or genomic DNA using these oligonucleotide primers and standard PCR amplification techniques. The nucleic acid so amplified can be cloned into an appropriate vector and characterized by DNA sequence analysis. cDNA may be prepared from mRNA, by isolating total cellular mRNA by a variety of techniques, for example, by using the guanidinium-thiocyanate extraction procedure of Chirgwin et al., Biochemistry, 18,5294-5299 (1979). cDNA is then synthesized from the mRNA using reverse transcriptase (for example, Moloney MLV reverse transcriptase available from Gibco/BRL, Bethesda, Md., or AMV reverse transcriptase available from Seikagaku America, Inc., St. Petersburg, Fla.).

An isolated nucleic acid molecule of the invention which is RNA can be isolated by cloning a nucleic acid molecule of the invention which is cDNA into an appropriate vector which allows for transcription of the cDNA to produce an RNA molecule. For example, a cDNA can be cloned downstream of a bacteriophage promoter, (e.g. a T7 promoter) in a vector, cDNA can be transcribed in vitro with T7 polymerase, and the resultant RNA can be isolated by conventional techniques.

Nucleic acid molecules of the invention may be chemically synthesized using standard techniques. Methods of chemically synthesizing polydeoxynucleotides are known, including but not limited to solid-phase synthesis which, like peptide synthesis, has been fully automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071).

Determination of whether a particular nucleic acid molecule encodes a protein of the invention can be accomplished by expressing the cDNA in an appropriate host cell by standard techniques, and testing the expressed protein using conventional methods. A cDNA having the biological activity of a protein of the invention can be sequenced by standard techniques, such as dideoxynucleotide chain termination or Maxam-Gilbert chemical sequencing, to determine the nucleic acid sequence and the predicted amino acid sequence of the encoded protein.

The initiation codon and untranslated sequences of a nucleic acid molecule of the invention may be determined using computer software designed for the purpose, such as PC/Gene (IntelliGenetics Inc., Calif.). The intron-exon structure and the transcription regulatory sequences of a nucleic acid molecule or gene of the invention may be identified by using a nucleic acid molecule of the invention to probe a genomic DNA clone library. Regulatory elements can be identified using standard techniques. The function of the elements can be confirmed by using these elements to express a reporter gene such as the lacZ gene which is operatively linked to the elements. These constructs may be introduced into cultured cells using conventional procedures or into non-human transgenic animal models. In addition to identifying regulatory elements in DNA, such constructs may also be used to identify nuclear proteins interacting with the elements, using techniques known in the art.

The invention contemplates polynucleotides comprising all or a portion of a nucleic acid of the invention comprising a regulatory sequence of a nucleic acid molecule of the invention contained in appropriate expression vectors. The vectors may contain sequences encoding heterologous proteins.

In accordance with another aspect of the invention, the nucleic acids isolated using the methods described herein are mutant gene alleles. For example, the mutant alleles may be isolated from individuals either known or proposed to have a genotype which contributes to the symptoms of a condition affecting hematopoiesis etc. Mutant alleles and mutant allele products may be used in therapeutic and diagnostic methods described herein. For example, a cDNA of a mutant gene may be isolated using PCR as described herein, and the DNA sequence of the mutant allele may be compared to the normal allele to ascertain the mutation(s) responsible for the loss or alteration of function of the mutant gene product. A genomic library can also be constructed using DNA from an individual suspected of or known to carry a mutant allele, or a cDNA library can be constructed using RNA from tissue known, or suspected to express the mutant allele. A nucleic acid encoding a normal gene or any suitable fragment thereof, may then be labeled and used as a probe to identify the corresponding mutant allele in such libraries. Clones containing mutant sequences can be purified and subjected to sequence analysis. In addition, an expression library can be constructed using cDNA from RNA isolated from a tissue of an individual known or suspected to express a mutant allele. Gene products made by the putatively mutant tissue may be expressed and screened, for example using antibodies specific for a protein of the invention as described herein. Library clones identified using the antibodies can be purified and subjected to sequence analysis.

The sequence of a nucleic acid molecule of the invention may be inverted relative to its normal presentation for transcription to produce an antisense nucleic acid molecule. An antisense nucleic acid molecule may be constructed using chemical synthesis and enzymatic ligation reactions using procedures known in the art.

2.2 Proteins of the Invention

The proteins of the invention are primarily expressed in hematopoietic, endothelial, stromal, and/or myocyte lineages. Amino acid sequences of proteins of the invention comprise the sequences of SEQ. ID. NO.2, SEQ. ID. NO 5., or SEQ. ID. NO. 7.

In addition to the amino acid sequences as shown SEQ. ID. NO.2, SEQ. ID. NO 5., or SEQ. ID. NO. 7, the proteins of the present invention include truncations of the proteins of the invention, and analogs, and homologs of the proteins and truncations thereof as described herein. Truncated proteins may comprise peptides of between 3 and 275 amino acid residues, ranging in size from a tripeptide to a 275 mer polypeptide.

The truncated proteins may have an amino group (—NH2), a hydrophobic group (for example, carbobenzoxyl, dansyl, or T-butyloxycarbonyl), an acetyl group, a 9-fluorenylmethoxy-carbonyl (PMOC) group, or a macromolecule including but not limited to lipid-fatty acid conjugates, polyethylene glycol, or carbohydrates at the amino terminal end. The truncated proteins may have a carboxyl group, an amido group, a T-butyloxycarbonyl group, or a macromolecule including but not limited to lipid-fatty acid conjugates, polyethylene glycol, or carbohydrates at the carboxy terminal end.

The proteins of the invention may also include analogs, and/or truncations thereof as described herein, which may include, but are not limited to the proteins, containing one or more amino acid substitutions, insertions, and/or deletions. Amino acid substitutions may be of a conserved or non-conserved nature. Conserved amino acid substitutions involve replacing one or more amino acids with amino acids of similar charge, size, and/or hydrophobicity characteristics. When only conserved substitutions are made the resulting analog should be functionally equivalent to the native protein. Non-conserved substitutions involve replacing one or more amino acids with one or more amino acids which possess dissimilar charge, size, and/or hydrophobicity characteristics.

One or more amino acid insertions may be introduced into a protein of the invention. Amino acid insertions may consist of single amino acid residues or sequential amino acids ranging from 2 to 15 amino acids in length.

Deletions may consist of the removal of one or more amino acids, or discrete portions from the protein sequence. The deleted amino acids may or may not be contiguous. The lower limit length of the resulting analog with a deletion mutation is about 10 amino acids, preferably 100 amino acids.

An allelic variant at the protein level differs from another protein by only one, or at most, a few amino acid substitutions. A species variation of a protein of the invention is a variation which is naturally occurring among different species of an organism.

The proteins of the invention also include homologs and/or truncations thereof as described herein. Such homologs include proteins whose amino acid sequences are comprised of the amino acid sequences of regions from other species that hybridize under selective hybridization conditions (see discussion of selective and in particular stringent hybridization conditions herein) with a probe used to obtain a protein of the invention. These homologs will generally have the same regions which are characteristic of a protein of the invention. It is anticipated that a protein comprising an amino acid sequence which is at least 75% identical, preferably 80 to 90% identical, with an amino acid sequence of SEQ. ID. NO.2, SEQ. ID. NO 5., or SEQ. ID. NO. 7 will be a homolog.

A percent amino acid sequence homology or identity is calculated as the percentage of aligned amino acids that match the reference sequence, where the sequence alignment has been determined using the alignment algorithm of Dayhoff et al; Methods in Enzymology 91: 524-545 (1983).

The invention also contemplates isoforms of the proteins of the invention. An isoform contains the same number and kinds of amino acids as the protein of the invention, but the isoform has a different molecular structure. The isoforms contemplated by the present invention are those having the same properties as a protein of the invention as described herein.

The present invention also includes proteins of the invention conjugated with a selected protein, or a selectable marker protein (see below) to produce fusion proteins. Additionally, immunogenic portions of a protein of the invention are within the scope of the invention.

A protein of the invention may be prepared using recombinant DNA methods. Accordingly, the nucleic acid molecules of the present invention having a sequence which encodes a protein of the invention may be incorporated in a known manner into an appropriate expression vector which ensures good expression of the protein. Possible expression vectors include but are not limited to cosmids, plasmids, or modified viruses (e.g. replication defective retroviruses, adenoviruses and adeno-associated viruses), so long as the vector is compatible with the host cell used.

The invention therefore contemplates a vector of the invention containing a nucleic acid molecule of the invention, and optionally the necessary regulatory sequences for the transcription and translation of the inserted protein-sequence. Suitable regulatory sequences may be derived from a variety of sources, including bacterial, fungal, viral, mammalian, or insect genes (For example, see the regulatory sequences described in Goeddel, Gene Expression Technology: Methods in Enzymology 185, Academic Press, San Diego, Calif. (1990). Selection of appropriate regulatory sequences is dependent on the host cell chosen as discussed below, and may be readily accomplished by one of ordinary skill in the art. The necessary regulatory sequences may be supplied by a native protein and/or its flanking regions.

The invention further provides a vector comprising a DNA nucleic acid molecule of the invention cloned into the vector in an antisense orientation. That is, the DNA molecule is linked to a regulatory sequence in a manner which allows for expression, by transcription of the DNA molecule, of an RNA molecule which is antisense to a nucleic acid sequence of a nucleic acid molecule of the invention. Regulatory sequences linked to the antisense nucleic acid can be chosen which direct the continuous expression of the antisense RNA molecule in a variety of cell types, for instance a viral promoter and/or enhancer, or regulatory sequences can be chosen which direct tissue or cell type specific expression of antisense RNA.

The expression vector of the invention may also contain a selectable marker gene which facilitates the selection of host cells transformed or transfected with a vector of the invention. Examples of selectable marker genes are genes encoding a protein such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. The selectable markers can be introduced on a separate vector from the nucleic acid of interest.

The vectors may also contain genes which encode a fusion moiety which provides increased expression of the recombinant protein; increased solubility of the recombinant protein; and aid in the purification of the target recombinant protein by acting as a ligand in affinity purification. For example, a proteolytic cleavage site may be added to the target recombinant protein to allow separation of the recombinant protein from the fusion moiety subsequent to purification of the fusion protein. Typical fusion expression vectors include pGEX (Amrad Corp., Melbourne, Australia), pMAL (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

The vectors may be introduced into host cells to produce a transformant host cell. “Transformant host cells” include host cells which have been transformed or transfected with a vector of the invention. The terms “transformed with”, “transfected with”, “transformation” and “transfection” encompass the introduction of nucleic acid (e.g. a vector) into a cell by one of many standard techniques. Prokaryotic cells can be transformed with nucleic acid by, for example, electroporation or calcium-chloride mediated transformation. Nucleic acid can be introduced into mammalian cells via conventional techniques such as calcium phosphate or calcium chloride co-precipitation, DEAE-dextran-mediated transfection, lipofectin, electroporation or microinjection. Suitable methods for transforming and transfecting host cells can be found in Sambrook et al. (Molecular Cloning: A Laboratory Manual, 2nd Edition, Cold Spring Harbor Laboratory press (1989)), and other laboratory textbooks.

Suitable host cells include a wide variety of prokaryotic and eukaryotic host cells. For example, the proteins of the invention may be expressed in bacterial cells such as E. coli, insect cells (using baculovirus), yeast cells, or mammalian cells. Other suitable host cells can be found in Goeddel, Gene Expression Technology: Methods in Enzymology 185, Academic Press, San Diego, Calif. (1991).

A host cell may also be chosen which modulates the expression of an inserted nucleic acid sequence, or modifies (e.g. glycosylation or phosphorylation) and processes (e.g. cleaves) the protein in a desired fashion. Host systems or cell lines may be selected which have specific and characteristic mechanisms for post-translational processing and modification of proteins. For example, eukaryotic host cells including CHO, VERO, BHK, HeLA, COS, MDCK, 293, 3T3, and WI38 may be used. For long-term high-yield stable expression of the protein, cell lines and host systems which stably express the gene product may be engineered.

Host cells and in particular cell lines produced using the methods described herein may be particularly useful in screening and evaluating compounds that modulate the activity of a protein of the invention.

The proteins of the invention may also be expressed in non-human transgenic animals including but not limited to mice, rats, rabbits, guinea pigs, micro-pigs, goats, sheep, pigs, non-human primates (e.g. baboons, monkeys, and chimpanzees) (see Hammer et al. (Nature 315:680-683, 1985), Palmiter et al. (Science 222:809-814, 1983), Brinster et al. (Proc Natl. Acad. Sci USA 82:44384442, 1985), Palmiter and Brinster (Cell. 41:343-345, 1985) and U.S. Pat. No. 4,736,866). Procedures known in the art may be used to introduce a nucleic acid molecule of the invention encoding a protein of the invention into animals to produce the founder lines of transgenic animals. Such procedures include pronuclear microinjection, retrovirus mediated gene transfer into germ lines, gene targeting in embryonic stem cells, electroporation of embryos, and sperm-mediated gene transfer.

The present invention contemplates a transgenic animal that carries a nucleic acid molecule of the invention in all their cells, and animals which carry the transgene in some but not all their cells. The transgene may be integrated as a single transgene or in concatamers. The transgene may be selectively introduced into and activated in specific cell types (See for example, Lasko et al, 1992 Proc. Natl. Acad. Sci. USA 89: 6236). The transgene may be integrated into the chromosomal site of the endogenous gene by gene targeting. The transgene may be selectively introduced into a particular cell type inactivating the endogenous gene in that cell type (See Gu et al Science 265: 103-106).

The expression of a recombinant protein of the invention in a transgenic animal may be assayed using standard techniques. Initial screening may be conducted by Southern Blot analysis, or PCR methods to analyze whether the transgene has been integrated. The level of mRNA expression in the tissues of transgenic animals may also be assessed using techniques including Northern blot analysis of tissue samples, in situ hybridization, and RT-PCR. Tissue may also be evaluated immunocytochemically using antibodies against GNTV Protein.

The proteins of the invention may also be prepared by chemical synthesis using techniques well known in the chemistry of proteins such as solid phase synthesis (Merrifield, 1964, J. Am. Chem. Assoc. 85:2149-2154) or synthesis in homogenous solution (Houbenweyl, 1987, Methods of Organic Chemistry, ed. E. Wansch, Vol. 15 I and II, Thieme, Stuttgart).

N-terminal or C-terminal fusion proteins comprising a protein of the invention conjugated with other molecules, such as proteins may be prepared by fusing, through recombinant techniques, the N-terminal or C-terminal of a protein of the invention, and the sequence of a selected protein or selectable marker protein with a desired biological function. The resultant fusion proteins contain a protein of the invention fused to the selected protein or marker protein as described herein. Examples of proteins which may be used to prepare fusion proteins include immunoglobulins, glutathione-S-transferase (GST), hemagglutinin (HA), and truncated myc.

2.3 Nucleotide Probes

The nucleic acid molecules of the invention allow those skilled in the art to construct nucleotide probes for use in the detection of nucleic acid sequences in biological materials. Suitable probes include nucleic acid molecules based on nucleic acid sequences of the invention and in particular nucleic acid sequences encoding at least 6 sequential amino acids from regions of a protein of the invention (e.g SEQ. ID. NO.2, SEQ. ID. NO 5., or SEQ. ID. NO. 7). A nucleotide probe may be labelled with a detectable substance such as a radioactive label which provides for an adequate signal and has sufficient half-life such as ³²P, ³H, ¹⁴C or the like. Other detectable substances which may be used include antigens that are recognized by a specific labelled antibody, fluorescent compounds, enzymes, antibodies specific for a labelled antigen, and luminescent compounds. An appropriate label may be selected having regard to the rate of hybridization and binding of the probe to the nucleotide to be detected and the amount of nucleotide available for hybridization. Labelled probes may be hybridized to nucleic acids on solid supports such as nitrocellulose filters or nylon membranes as generally described in Sambrook et al, 1989, Molecular Cloning, A Laboratory Manual (2nd ed.).

The nucleotide probes may also be useful in the diagnosis of disorders of the hematopoietic system, sensory nervous system, myocardium, or cardiac or neural vasculature, in monitoring the progression of these conditions; or monitoring a therapeutic treatment.

A probe may be used in hybridization techniques to detect nucleic acid molecules or genes of the invention. The technique generally involves contacting and incubating nucleic acids obtained from a sample from a patient or other cellular source with a probe of the present invention under conditions favourable for the specific annealing of the probes to complementary sequences in the nucleic acids. After incubation, the non-annealed nucleic acids are removed, and the presence of nucleic acids that have hybridized to the probe if any are detected.

The detection of nucleic acid molecules of the invention may involve the amplification of specific gene sequences using an amplification method such as PCR, followed by the analysis of the amplified molecules using techniques known to those skilled in the art. Suitable primers can be routinely designed by one of skill in the art.

Genomic DNA may be used in hybridization or amplification assays of biological samples to detect abnormalities in a gene or nucleic acid molecule of the invention, including point mutations, insertions, deletions, and chromosomal rearrangements. For example, direct sequencing, single stranded conformational polymorphism analyses, heteroduplex analysis, denaturing gradient gel electrophoresis, chemical mismatch cleavage, and oligonucleotide hybridization may be utilized.

Genotyping techniques known to one skilled in the art can be used to type polymorphisms that are in close proximity to mutations in a nucleic acid molecule or gene of the invention. The polymorphisms may be used to identify individuals in families that are likely to carry mutations. If a polymorphism exhibits linkage disequalibrium with mutations in a gene, it can also be used to screen for individuals in the general population likely to carry mutations. Polymorphisms which may be used include restriction fragment length polymorphisms (RFLPs), single-base polymorphisms, and simple sequence repeat polymorphisms (SSLPs).

A probe of the invention may be used to directly identify RFLPs. A probe or primer of the invention can additionally be used to isolate genomic clones such as YACs, BACs, PACs, cosmids, phage or plasmids. The DNA in the clones can be screened for SSLPs using hybridization or sequencing procedures.

Hybridization and amplification techniques described herein may be used to assay qualitative and quantitative aspects of expression of a nucleic acid molecule of the invention. For example, RNA may be isolated from a cell type or tissue known to express a gene and tested utilizing the hybridization (e.g. standard Northern analyses) or PCR techniques referred to herein. The techniques may be used to detect differences in transcript size which may be due to normal or abnormal alternative splicing. The techniques may be used to detect quantitative differences between levels of full length and/or alternatively splice transcripts detected in normal individuals relative to those individuals exhibiting symptoms of a disease.

The primers and probes may be used in the above described methods in situ i.e directly on tissue sections (fixed and/or frozen) of patient tissue obtained from biopsies or resections.

2.4 Antibodies

Proteins of the invention can be used to prepare antibodies specific for the proteins. Antibodies can be prepared which bind a distinct epitope in an unconserved region of the protein. An unconserved region of the protein is one which does not have substantial sequence homology to other proteins. A region from a well-characterized region can be used to prepare an antibody to a conserved region of a protein of the invention. Antibodies having specificity for a protein of the invention may also be raised from fusion proteins created by expressing fusion proteins in bacteria as described herein.

The invention can employ intact monoclonal or polyclonal antibodies, and immunologically active fragments (e.g. a Fab or (Fab)₂ fragment), an antibody heavy chain, and antibody light chain, a genetically engineered single chain Fᵥ molecule (Ladner et al, U.S. Pat. No. 4.946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Antibodies specifically reactive with a protein of the invention, or derivatives, such as enzyme conjugates or labeled derivatives, may be used to detect the proteins in various biological materials, for example they may be used in any known immunoassays which rely on the binding interaction between an antigenic determinant of a protein and the antibodies. Examples of such assays are radioimmunoassays, enzyme immunoassays (e.g.ELISA), immunofluorescence, immunoprecipitation, latex agglutination, hemagglutination, and histochemical tests. The antibodies may be used to detect and quantify a protein of the invention in a sample in order to determine its role in particular cellular events or pathological states, and to diagnose and treat such pathological states.

In particular, the antibodies of the invention may be used in immuno-histochemical analyses, for example, at the cellular and sub-subcellular level, to detect a protein of the invention, to localise it to particular cells and tissues, and to specific subcellular locations, and to quantitate the level of expression.

Cytochemical techniques known in the art for localizing antigens using light and electron microscopy may be used to detect a protein of the invention. Generally, an antibody of the invention may be labelled with a detectable substance and a protein may be localised in tissues and cells based upon the presence of the detectable substance. Examples of detectable substances include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol; enzymatic labels (e.g., horseradish peroxidase, .beta.-galactosidase, luciferase, alkaline phosphatase, acetylcholinesterase), biotinyl groups (which can be detected by marked avidin e.g., streptavidin containing a fluorescent marker or enzymatic activity that can be detected by optical or calorimetric methods), predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

Indirect methods may also be employed in which the primary antigen-antibody reaction is amplified by the introduction of a second antibody, having specificity for the antibody reactive against a protein of the invention. By way of example, if the antibody having specificity against a protein of the invention is a rabbit IgG antibody, the second antibody may be goat anti-rabbit gamma-globulin labelled with a detectable substance as described herein.

Where a radioactive label is used as a detectable substance, a protein of the invention may be localized by radioautography. The results of radioautography may be quantitated by determining the density of particles in the radioautographs by various optical methods, or by counting the grains.

2.5 Applications of the Nucleic Acid Molecules and Proteins of the Invention

The proteins of the invention are primarily expressed in hematopoietic, endothelial stromal, and/or myocyte lineages. The proteins of the invention have a role in proliferation, differentiation, activation and/or metabolism of cells of the hematopoietic, myocardium, cardiac and neural vasculature, endothelial, stromal, and/or myocyte lineages. Therefore, the methods described herein for detecting nucleic acid molecules can be used to monitor proliferation, differentiation, activation and/or metabolism of cells of the hematopoietic, endothelial, myocardium, cardiac and neural vasculature, stromal, and/or myocyte lineages by detecting and localizing proteins and nucleic acid molecules of the invention. The methods described herein may be used to study the developmental expression of a protein of the invention and, accordingly, will provide further insight into the role of the protein in the hematopoietic system, myocardium, sensory nervous system and vasculature.

By way of example, the 17G2 protein is expressed in the myocardium, cardiac and neural vasculature, in hematopoietic cells, and in the sensory nervous system. Therefore, the 17G2 protein has a role in proliferation, differentiation, activation and metabolism of cells of the hematopoietic system, myocardium, cardiac and neural vasculature, and the sensory nervous system. Therefore, the methods for detecting nucleic acid molecules and 17G2 proteins of the invention, can be used to monitor proliferation, differentiation, activation and metabolism of hematopoietic cells, and cells of the sensory nervous system and neural and cardiac vasculature by detecting and localizing 17G2 proteins and nucleic acid molecules. It would also be apparent to one skilled in the art that the above described methods may be used to study the developmental expression of 17G2 proteins and, accordingly, will provide further insight into the role of 17G2 proteins in the hematopoietic system, myocardium, neural and cardiac vasculature, and sensory nervous system.

The nucleic acid molecules and proteins of the invention are markers for hematopoietic cells, endothelial cells, stromal cells, and/or myocytes, and accordingly the antibodies and probes described herein may be used to label these cells. For example, the 17G2 protein is a marker for early vascular endothelial cells and hematopoietic cells, and accordingly the antibodies and probes described herein can be used to label early vascular endothelial cells and hematopoietic cells.

Substances which modulate a protein of the invention (e.g. a 17G2 protein) can be identified based on their ability to bind to the protein. Therefore, the invention also provides methods for identifying substances which bind to a protein of the invention. Substances identified using the methods of the invention may be isolated, cloned and sequenced using conventional techniques.

Substances which can bind with a protein of the invention e.g. a 17G2 protein may be identified by reacting the protein with a substance which potentially binds to the protein, under conditions which permit the formation of substance-protein complexes and assaying for substance-protein complexes, for free substance, for non-complexed protein, or for activated protein. Conditions which permit the formation of complexes may be selected having regard to factors such as the nature and amounts of the substance and the protein.

The substance-protein complex, free substance or non-complexed proteins may be isolated by conventional isolation techniques, for example, salting out, chromatography, electrophoresis, gel filtration, fractionation, absorption, polyacrylamide gel electrophoresis, agglutination, or combinations thereof. To facilitate the assay of the components, antibody against the protein or the substance, or labelled protein, or a labelled substance may be utilized. The antibodies, proteins, or substances may be labelled with a detectable substance as described above.

A protein, or the substance used in the method of the invention may be insolubilized. For example, the protein, or substance may be bound to a suitable carrier such as agarose, cellulose, dextran, Sephadex, Sepharose, carboxymethyl cellulose polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, etc. The carrier may be in the shape of, for example, a tube, test plate, beads, disc, sphere etc. The insolubilized protein or substance may be prepared by reacting the material with a suitable insoluble carrier using known chemical or physical methods, for example, cyanogen bromide coupling.

The invention also contemplates a method for evaluating a compound for its ability to modulate the biological activity of a protein of the invention, by assaying for an agonist or antagonist (i.e. enhancer or inhibitor) of the binding of the protein with a substance which binds with the protein. The enhancer or inhibitor may be an endogenous physiological compound or it may be a natural or synthetic compound.

It will be understood that the agonists and antagonists i.e. inhibitors and enhancers that can be assayed using the methods of the invention may act on one or more of the binding sites on the protein or substance including agonist binding sites, competitive antagonist binding sites, non-competitive antagonist binding sites or allosteric sites.

The invention also makes it possible to screen for antagonists that inhibit the effects of an agonist of the interaction of the protein with a substance which is capable of binding to the protein. Thus, the invention may be used to assay for a compound that competes for the same binding site of the protein.

The reagents suitable for applying the methods of the invention to evaluate compounds that modulate a protein of the invention may be packaged into convenient kits providing the necessary materials packaged into suitable containers. The kits may also include suitable supports useful in performing the methods of the invention.

The substances or compounds identified by the methods described herein, antibodies, and antisense nucleic acid molecules of the invention may be used for modulating the biological activity of a protein of the invention, and they may be used in the treatment of conditions requiring modulation of cells of the hematopoietic, myocardium, cardiac and neural vasculature, endothelial, stromal, and/or myocyte lineages. Accordingly, the substances, antibodies, and compounds may be formulated into pharmaceutical compositions for administration to subjects in a biologically compatible form suitable for administration in vivo. By “biologically compatible form suitable for administration in vivo” is meant a form of the substance to be administered in which any toxic effects are outweighed by the therapeutic effects. The substances may be administered to living organisms including humans, and animals. Administration of a therapeutically active amount of the pharmaceutical compositions of the present invention is defined as an amount effective, at dosages and for periods of time necessary to achieve the desired result. For example, a therapeutically active amount of a substance may vary according to factors such as the disease state, age, sex, and weight of the individual, and the ability of antibody to elicit a desired response in the individual. Dosage regima may be adjusted to provide the optimum therapeutic response. For example, several divided doses may be administered daily or the dose may be proportionally reduced as indicated by the exigencies of the therapeutic situation.

The active substance may be administered in a convenient manner such as by injection (subcutaneous, intravenous, etc.), oral administration, inhalation, transdermal application, or rectal administration. Depending on the route of administration, the active substance may be coated in a material to protect the compound from the action of enzymes, acids and other natural conditions which may inactivate the compound.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the substances or compounds in association with one or more pharmaceutically acceptable vehicles or diluents, and contained in buffered solutions with a suitable pH and iso-osmotic with the physiological fluids.

The activity of the substances, compounds, antibodies, antisense nucleic acid molecules, and compositions of the invention may be confirmed in animal experimental model systems.

The invention also provides methods for studying the function of a protein of the invention. Cells, tissues, and non-human animals lacking in expression or partially lacking in expression of a nucleic acid molecule or gene of the invention may be developed using recombinant expression vectors of the invention having specific deletion or insertion mutations in the gene. A recombinant expression vector may be used to inactivate or alter the endogenous gene by homologous recombination, and thereby create a deficient cell, tissue or animal.

Null alleles may be generated in cells, such as embryonic stem cells by deletion mutation. A recombinant gene may also be engineered to contain an insertion mutation which inactivates the gene. Such a construct may then be introduced into a cell, such as an embryonic stem cell, by a technique such as transfection, electroporation, injection etc. Cells lacking an intact gene may then be identified, for example by Southern blotting, Northern Blotting or by assaying for expression of the encoded protein using the methods described herein. Such cells may then be fused to embryonic stem cells to generate transgenic non-human animals deficient in a protein of the invention. Germline transmission of the mutation may be achieved, for example, by aggregating the embryonic stem cells with early stage embryos, such as 8 cell embryos, in vitro; transferring the resulting blastocysts into recipient females and; generating germline transmission of the resulting aggregation chimeras. Such a mutant animal may be used to define specific cell populations, developmental patterns and in vivo processes, normally dependent on gene expression.

The following non-limiting examples are illustrative of the present invention:

### EXAMPLES

### Example 1

Materials and Methods

Vectors. Two gene trap vectors were used. PT1-ATG (PT1 henceforth) contains the En-2 splice acceptor site positioned immediately upstream of the lacZ reporter gene with an ATG translational start site [Hill D. P., Wurst W., Methods in Enzymology 225:664-681, 1993]. The bacterial neomycin-resistance (neo) gene is driven by the phosphoglycerate kinase-1 (PGK-1) promoter. GT1.8geo contains the En-2 splice acceptor site immediately upstream of a lacZ-neo fusion gene [Skarnes W. C. et al, Proc. Natl. Acad. Sci. USA 92:6592-6596, 1995]. The point mutation in the neo fragment of SAβgeo is not contained in GT1.8geo vector, thereby allowing neomycin resistance at a lower level of endogenous gene expression than the SAβgeo vector. Generation of Trapped ES Cell Lines. R1 ES cells were maintained on primary embryonic fibroblasts as previously described [Nagy A. et al., Proc. Natl. Acad. Sci. USA 90:8424-8428, 1993]. After electroporation and selection in G418 for 8 days, drug-resistant colonies were transferred to 96-well plates and expanded to confluency. Clones were passaged to two 96-well plates and one set of 24-well plates. Once clones reached confluency, one 96-well plate was frozen, the second 96-well plate was assayed for β-galactosidase (β-gal) expression, and the 24-well plates were used for attached EB differentiation cultures. Expression of the lacZ reporter gene was carefully determined both in undifferentiated and differentiated ES cells. Clones with observable expression patterns were re-frozen and in some cases, re-analyzed. In addition, the expression patterns were photographed and cataloged. Reporter Gene Expression. β-gal activity of undifferentiated and differentiated cells was detected as follows: Cells were rinsed in 100 mM sodium phosphate (pH 7.5), then fixed in 0.2% glutaraldehyde, 5 mM EGTA, 2 mM MgCl₂ and 100 mM sodium phosphate, pH 7.5 for 5 min. The cells were washed 3 times for 5 min. each in 2 mM MgCl₂, 0.02% NP-40 and 100 mM sodium phosphate, pH 7.5. The cells were stained with X-gal overnight at 37° C. β-gal activity was detected in embryos as described above except the fixative included 1.5% formaldehyde and embryos were fixed for 30 min. to 1 hour and washed 3 times for 15 min. each wash. Attached EB Screen. ES cells were allowed to differentiate into attached EBs as previously described [Bautch V. L. et al., Dev. Dyn. 205:1-12, 1996] with several modifications. Clones were grown to confluency in 24-well plates, treated with dispase (Collaborative Research, 1:1 dilution in PBS), washed 3 times in PBS and grown in suspension in “Ultra Low Cluster” 24-well plates (COSTAR) in ES media without LIF. On day 3 post-dispase treatment, 5-10 embryoid bodies were transferred to 48-well tissue culture plates (Falcon). Cultures were fed every other day with fresh media. β-gal activity was determined on day 8, 12, and 16 post-dispase. OP9 Induction Assay. ES cells were allowed to differentiate on the OP9 stromal cell line as previously described [Nakano T. et al., Science 265:1098-1101, 1994] with several modifications. ES clones were differentiated on OP9 stroma in replica wells of 6-well plates (10⁴ ES cells/well) for 5 days to generate mesodermal colonies. A single cell suspension was prepared using trypsin from one well for each clone, and 10⁵ mesodermal cells were replated onto OP9 stroma in two wells of a 6-well plate and grown for 3 days. Non-adherent hematopoietic cells were transferred from both wells to one new well for an additional 3 days. β-gal activity was determined on mesodermal cells on the duplicate day 5 OP9 plate and on adherent hematopoietic cells on days 8 and 11. 5′ RACE. RNA was prepared from either undifferentiated or differentiated cells using Trizol (Gibco/BRL) according to manufacturer's instructions. 5′ RACE was performed using the 5′ RACE kit (Gibco/BRL), according to manufacturer's instructions with modifications previously described [Sam M. et al., Dev. Dyn., in press]. 5′ RACE products were subcloned into the CloneAmp plasmid (Gibco/BRL) and sequenced using the Sequenase kit (Pharmacia) according to manufacturers' instructions. Sequences were analyzed by comparison to the non-redundant GenBank and EST of NCBI using the BLASTN program. Generation of Chimeras. ES cells were aggregated with diploid embryos as described [Nay A., Rossant, J., Oxford, IRL, 1993, p. 147-178], harvested at embryonic day (e) 9.5-14.5, and stained for β-gal activity. About half of the diploid embryos were allowed to mature to term for germ-line transmission. Chimeric males were bred to CD1 females, and tail DNA of F₁ and F₂ offspring was analyzed by southern blotting and hybridization to En-2 or RACE fragment probes.

Results

Identification of Trapped Gene Expression Patterns. In the absence of leukemic inhibitory factor, ES colonies spontaneously differentiate into embryoid bodies (EBs) in suspension culture. The complex structure of the EB contains all three germ layers and resembles the extra-embryonic yolk sac both morphologically and transcriptionally [Doetschmann T. C. et al., J. Embryol. Exp. Morph. 87:27-45, 1985], [Schmitt, R. M. et al., Genes & Dev. 5:728-740, 1991], [Keller G. et al., Mol. Cell. Biol. 13:473-486, 19931, [Snodgrass H. R. et al., American Association of Blood Banks, 1993, p 65-83]. As in the yolk sac, the mesoderm of the EB gives rise to angioblastic cords that form blood islands containing primitive hematopoietic cells surrounded by vascular endotheliumWang R. et al., Development 114:303-316, 1992]. Due to the developmental potential of EBs, the differentiation of ES cells into EBs has provided an excellent model to study the effects of targeted mutations on hematopoietic, vascular and myoblast lineages [Weiss M. J. et al., Genes & Dev. 8:1184-1197, 1994, Shalaby F. et al., Cell 89:981-990, 1997, Narita N. et al., Development 122:3755-3764 1996]. However, EBs grown in suspension are difficult to manipulate in clonal cultures and the outer layer of visceral endoderm precludes the identification of small numbers of lacZ positive cells. Therefore, the EB culture system was modified so that EBs grow attached to tissue culture plastic [Bautch V. L. et al., Dev. Dyn. 205:1-12, 1996]. This “attached” or “flat” culture method places the endoderm layer beneath the blood islands and renders the EB more accessible to observation and experimental manipulation.

The PT1 gene trap vector, which contains a splice acceptor site immediately upstream of a promoterless lacZ reporter gene and the neo gene driven by PGK-1 promoter, was introduced into ES cells (clone R1) by electroporation. After G418 selection, drug-resistant colonies were transferred to 96-well plates and expanded to confluency. Clones were replica plated to two 96-well plates and one set of 24-well plates. Once clones reached confluency, one 96-well plate was frozen, the second 96-well plate was assayed for β-galactosidase (β-gal) expression, and the 24-well plates were used for attached EB differentiation cultures. Each neo^{R} colony represented a vector integration event. If the vector integrated within an intron, a spliced fusion transcript between lacZ and the endogenous gene was generated upon transcriptional activation of the trapped gene. Because all ES cells which had an integrated PT1 vector were G418 resistant regardless of whether or not the integration occurred within a gene, genes which were not expressed in undifferentiated ES cells could be screened using this vector. Five percent (37/779) of the neo^{R} clones tested expressed lacZ in undifferentiated ES cells, of which 30 clones continued to be expressed in at least some cells during EB differentiation (Table 1). By comparison, 61 clones (8%) which did not express lacZ as undifferentiated ES cells demonstrated lacZ expression during EB differentiation (Table 1). Of the neo^{R} clones that expressed lacZ as undifferentiated or differentiated ES cells, one-third (32 clones) exhibited a restricted pattern of expression (Table 1). The expression patterns of these clones can be grouped into seven categories (Table 2). More than a third of the clones were expressed in blood islands and/or the vasculature; in contrast, stromal and muscle cells each represented only 3% of the clones displaying restricted expression patterns. In addition, 9% of the clones expressed lacZ constitutively in virtually all undifferentiated and differentiated cells. The remaining clones exhibited restricted patterns of expression in other cell type(s).

In a second series of experiments, the GT1.8geo vector which contains a splice-acceptor site immediately upstream of a β-gal-neo fusion gene was used. Thus, unlike the PT1 vector, all neo^{R} clones selected after introduction of the GT1.8geo vector represented integrations into genes which were transcriptionally active in undifferentiated ES cells. Accordingly, a much higher proportion of the GT1.8geo clones (34% versus 5% for PT1) expressed detectable levels of β-gal activity in undifferentiated ES cells (i.e., “Blue”, Table 1). Of those, 159 clones continued to express lacZ in at least some cells during EB differentiation. Of the clones which were lacZ negative as undifferentiated ES cells, more than half upregulated expression of lacZ in a portion of differentiated cells in EB cultures. In total, 47 clones displayed an obvious pattern of expression (Table 1 and 2). The majority of the pattern-expressing clones expressed lacZ in the blood islands and/or the endothelium (Table 2)

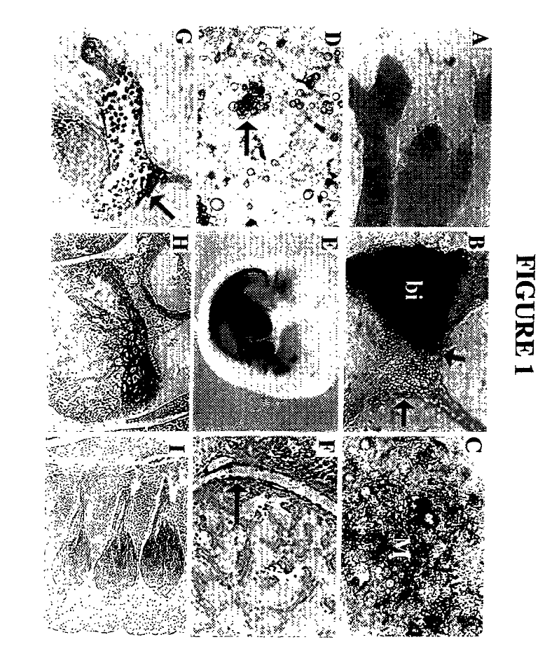

In contrast to EB body differentiation in which ES cells differentiate into all three germ layers which eventually give rise to many lineages including hematopoictic and vascular cells, ES cells grown in co-culture with OP9 stromal cells differentiate into mesodermal colonies which when replaced differentiate into hematopoietic cells. All gene trap cell lines demonstrating lacZ expression in blood islands were re-analyzed by differentiating ES cells in replicate OP9 stromal cell cultures[Nakano T. et al., Science 265:1098-1101, 1994], [Nakano T. et al., Science 272:722-724, 1996]. ES-derived mesodermal colonies expressing brachury were apparent by day 3 of culture. On day 5, a single cell suspension of a replicate culture was prepared and replated onto OP9 cells. Primitive erythrocytes and multipotential precursors differentiated from the mesodermal precursors within the next 2-3 days and single lineage precursors predominated the cultures by day 11. Cultures were assayed for lacZ expression at days 5, 8, and 11. The majority of blood island positive clones (70%) expressed lacZ in hematopoietic cells when cultured on an OP9 feeder layer (Table 2). Identification of Trapped Genes. To determine the DNA sequence of the trapped genes, RNA was prepared from either differentiated or undifferentiated ES clones and used to perform 5′ RACE [Frohman M. A. et al., Proc. Natl. Acad. Sci. USA 85:8998-9002, 1988]. The RACE products of eleven lacZ fusion transcripts were cloned and sequenced. Table 3 summarizes the lacZ expression pattern, the gene trap vector, and sequence information for each clone. Eight of the RACE product sequences corresponded to novel genes, of which four shared similarity with EST sequences. The sequences of three of the trapped genes corresponded to genes that encode known protein products: Mena, Karyopherin β3, and 5′GMP synthetase. Clone K18E2 encodes Mena, the mammalian homologue of Drosophilia Enabled(ena), which was originally cloned by a genetic screen for suppressors of Ab1-dependent phenotypes [Gertler F. B. et al., Genes & Dev. 9:521-533, 1995], [Gertler F. B. et al., Cell 87:227-239, 1996]. In clone K18E2, the PT1 vector has integrated into the first intron of Mena, downstream of the initiation codon and, therefore, should result in a null mutation. Clone B2C3 encodes the murine homologue of karyopherin/importin β3 and yeast Pse1p [Yaseen N. R., Blobel G., Proc. Natl. Acad. Sci. USA 94:4451-4456, 1997], proteins which are involved in the transport of proteins and mRNA across the nuclear membrane [Kutay U. et al., EMBO J. 16:1153-1163, 1997], [Seedorf M., Silver P. A., Proc. Natl. Acad. Sci. USA 94:8590-8595, 1997]. The RACE product suggests that a fusion protein was generated from the N-terminal 312 amino acids and lacZ. Mutational analysis of Xenopus karyopherin-α suggests that this fusion protein will bind weakly to the nuclear pore complex and to RanGTP but not to karyopherin-α [Kutay U. et al., EMBO J. 16: 1153-1163, 1997] and may act as a weak dominate negative mutation. In ES clone GC10G7, the GT1.8geo vector has integrated within the 3′ coding region of the gene for guanosine 5′-monophosphate (GMP) synthetase. GMP-synthetase catalyzes the amination of xanthosine 5′-monophosphate to form GMP in the presence of glutamine and ATP. Although GMP-synthetase is expressed in many cell types, high levels of β-gal activity were observed only in endothelial cells and a population of hematopoietic cells (Table 3). In Vitro and In Vivo Expression of Selected Clones. To determine if in vitro expression patterns correlated with in vivo expression, selected ES clones were aggregated with diploid embryos to generate chimeric mice. Reporter gene expression was performed first on chimeric embryos to quickly assess expression patterns and subsequently was confirmed in F₁ embryos, which is summarized along with sequence analysis in Table 1. Three clones corresponded to a sequence homolgous to an EST, a completely novel gene and Mena. K17G2 was isolated using the PT1 vector and displayed significant sequence similarity to a human EST. K17G2-lacZ was expressed at low to medium levels in undifferentiated ES cells (FIG. 1A), while its expression was restricted to blood islands and some endothelial cells in attached EBs (FIG. 1B). Differentiation on OP9 stromal cells revealed that K17G2-lacZ was expressed in some mesodermal and hematopoietic cells (FIG. 1C&D, respectively). To analyze the expression pattern of K17G2-lacZ in vivo, K17G2 ES cells were used to generate chimeric mice. Analysis of F₁ e10.5 embryos revealed additional tissues which expressed the K17G2-lacZ fusion product (FIG. 1E). For example, the lacZ fusion product was expressed in the myocardium and the dorsal root ganglia (FIG. 1F&G, respectively). However, as predicted by the in vitro expression, K17G2-lacZ was expressed in some of the embryonic vasculature, including the endocardium, and circulating blood cells (FIG. 1H&I). In the adult, K17G2-lacZ expression was observed in hematopoietic cells of the spleen and bone marrow and in the endocardium (data not shown). K17G2 heterozygous littermates were mated with one another; however, these matings failed to produce viable homozygous mice indicating that K17G2 homozygous embryos die in utero (data not shown).

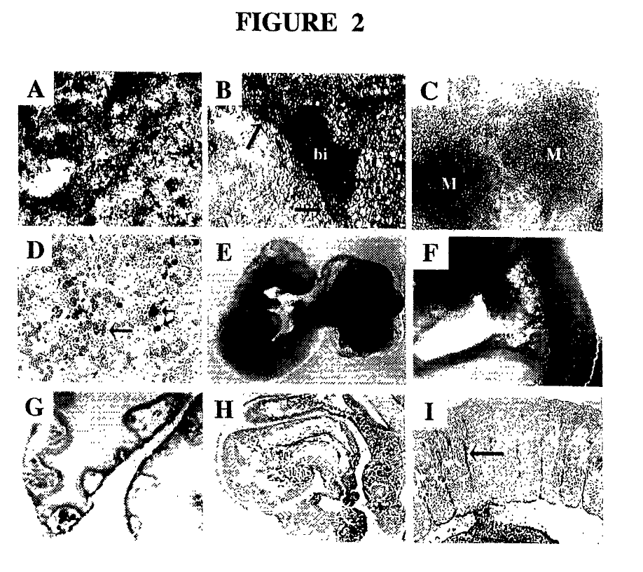

Clone GC11E10 was isolated using the GT1.8geo vector and represents a novel ORF. The GC11E10-geo fusion protein was expressed at medium to high levels in undifferentiated ES cells (FIG. 2A). In attached EBs, expression appeared within blood islands and the vasculature associated with these structures (FIG. 2B). Differentiation of GC11E10 ES cells on OP9 stromal cells demonstrated lacZ expression within mesodermal colonies and high levels of expression within hematopoietic cell clusters (FIG. 2C&D, respectively). In vivo, lacZ was expressed in the yolk sac, dorsal aorta, heart, the developing liver and vasculature (FIG. 2E&F). Further analysis demonstrated that lacZ expression was contained within blood cells circulating throughout the embryo and within blood islands in the yolk sac (FIG. 2G&H). The GC11E10-geo fusion protein was also expressed in endothelial cells throughout the embryo as demonstrated in the intersomitic vessels (FIG. 2I).

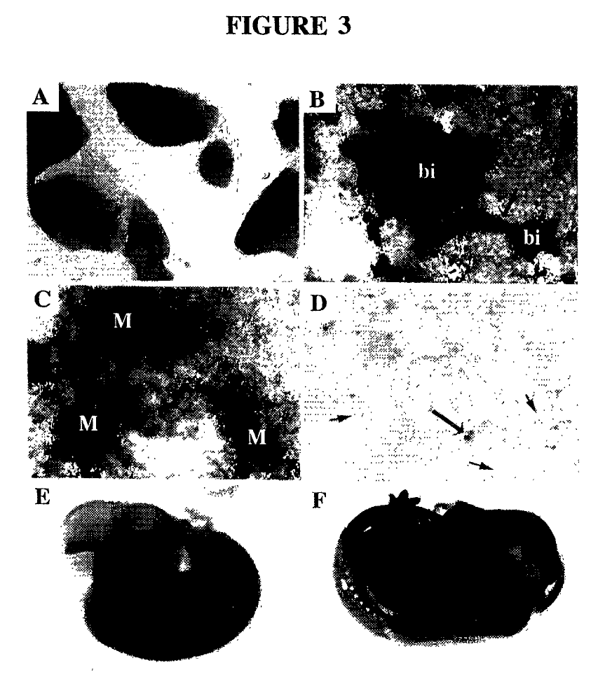

Clone K18E2 (a PT1 clone) represents an integration into the first intron of Mena. Mena is involved in actin assembly and cell motility; therefore its ubiquitous expression in rapidly dividing cells was expected. Mena-lacZ was expressed at very high levels in nearly all undifferentiated ES cells (FIG. 3A) and virtually all cells in EBs (FIG. 3B). Differentiation of K18E2 on OP9 stromal cells demonstrated high levels of Mena-lacZ expression in mesodermal cells (FIG. 4C) but only low level expression in a minority of hematopoietic cells (FIG. 4D). The pattern and level of lacZ expression was reproduced in F₁ embryos. Mena-lacZ was expressed by almost all cells in the developing embryo with the exception of hepatocytes and some hematopoietic cells (FIG. 4E&F and data not shown).

Discussion

The present inventors developed an expression-based strategy to identify and mutate genes that are preferentially expressed in cells of the hematopoietic and vascular lineages. Gene trap vectors were introduced into ES cells by electroporation and sibling clones were allowed to differentiate into attached EBs to identify expression patterns. Clones exhibiting reporter gene expression in blood islands were then differentiated on OP9 stromal cells to determine if hematopoietic cells expressed the reporter gene. From almost 1300 clones, 79 clones were isolated with identifiable expression patterns, of which 33 were preferentially expressed in hematopoietic and/or endothelial cells. These in vitro patterns of expression, which can be analyzed relatively quickly and in large numbers, were reliable predictors of in vivo expression patterns as determined in chimeric and F₁ embryos. ES clones with expression patterns of interest were then used to clone and sequence the upstream coding region of the trapped gene by 5′RACE. Three of the clones corresponded to known genes and eight were novel.

The attached EB differentiation assay used as the primary screen enabled the identification of a large number of genes with a spatially or cell-type restricted expression for several lineages including hematopoietic, endothelial, stromal and myocyte.

### Example 2

Gene trapping in embryonic stem (ES) cells coupled with two in vitro differentiation assays was used to screen for genes involved in hematopoietic and vascular development. Undifferentiated ES cells were electroporated with either the pPT1-ATG vector which contains a splice acceptor site upstream of a promoterless lac Z gene and a PGK-neoR gene, or the pGT1.8 geo vector which contains a promoterless lacZ/neoR fusion gene. G418 resistant clones were allowed to differentiate into attached embryoid bodies (EBs) and lacZ activity was assayed to indicate trapped gene expression in undifferentiated cells and differentiation cultures. Clones expressing lacZ in blood islands were also differentiated on OP9/OP9 stromal cells to confirm lacZ expression by hematopoietic cells.

A modified attached embryoid body (EB) assay was used to screen the reporter gene expression pattern of approximately 1300 gene trapped ES cell lines for expression in hematopoietic and endothelial lineages. The assay was carried out as described in V. L. Bautch et al., (Developmental Dynamics 205:1-12, 1996) with the following modifications. The ES clones were grown up in 24-well plates in the presence of lif (but without feeders) essentially as would be carried out in TC dishes. The media was aspirated, each well was washed with 1.5 ml PBS and aspirate. Cold diluted (1:1 IN PBS) Dispase was added to cover the well and it was allowed to sit 1-2 min at RT. The wells were filled with PBS and then pipetted up & down 2-3 times. The colonies were allowed to settle and the Dispase/PBS was aspirated or pipetted off. Washing was repeated with PBS, and using 1.5 ml CEB media. Clumps were transfered to 1.5 ml CEB media in wells of “Ultra Low Cluster 24 well plate” (COSTAR cat #3473). The plate was incubated at 37EC, 5%CO₂ for 3 days. On the third day post-Dispase, the embryoid bodies were pipetted up & down to mix, and about 2-4 drops were transferred into about 0.8 ml CEB media/well of a 48-well plate (Falcon cat #3078). The wells were checked to confirm that there were about 5 colonies/well. The plate was then incubated at 37EC, 5% CO₂ and the cultures were fed every other day.

The reporter gene expression pattern of clone 17G2 demonstrated moderate expression of the trapped gene in undifferentiated ES cells and restricted expression of hematopoietic and endothelial cells in the attached EB cultures. Differentiation of 17G2 on OP9 stromal cells lead to expression of the trapped gene in some mesodermal and hematopoietic cells. 17G2 ES cells were aggregated with wild-type CD1 embryos to generate chimeras. In vivo expression analysis reveals expression of the 17G2 gene in the cardiac and neural vasculature, hematopoietic cells, myocardium, and sensory nerves including the trigeminal ganglia, dorsal root ganglia, and optic nerve. 17G2 expression is maintained in the adult heart and bone marrow. The exon sequence upstream of the vector integration was cloned by 5′ RACE, and analysis showed that the 17G2 gene encodes a novel gene (see FIG. 1 for a nucleic acid sequence from the 17G2 gene). The RACE product was used as a probe to screen the genotypes of F₂ litters. No homozygotes were detected out of over 200 pups. Reporter gene expression analysis of timed heterozygous matings revealed that homozygous embryos are viable at midgestation (e11.5).

### Example 3

Analysis of 17G2 DNA sequence revealed that the cDNA sequence does not contain either the Kozak initiation sequence nor the termination and polyadenylation sequences. The 952 bp cDNA encodes a hydrophilic 317 amino acid open reading frame (ORF). The ORF contains numerous Protein Kinase C (PKC) and Casein Kinase II (CK2) phosphorylation sites as well as a tyrosine phosphorylation site. Comparison of the cDNA sequence to the non-redundant DNA databases revealed no significant matches. However, comparison of the cDNA to the EST databases using BLAST revealed six rat ESTs identified from subtractive libraries that were 97% identical to 17G2 and therefore are likely homologues to 17G2. In addition, a human EST, a Drosophilia EST, and a C.elegans full-length EST contiguous sequence encoding 466 amino acids were found to be 75%, 57%, and 50% identical, respectively. Amino acid comparison demonstrated 62% (66% conserved), 46% (68% conserved), and 40% (56% conserved) identical between 17G2 and the human EST, the C. elegans contig. sequence, and the Drosophilia EST, respectively. In addition, amino acid comparison by BLAST also demonstrated 30% and 42% identical and conserved, respectively with a yeast gene of unknown function termed yeast orf1. A more sophisticated amino acid analysis comparison program called Psi-BLAST determined that the 17G2 orf is similar (p=e−62) to the sorting nexins. Furthermore, the rat, human, C. elegans, Drosophilia, and yeast putative homologues of 17G2 as well as the sorting nexins all share the PKC, CK2, and tyrosine phosphorylation sites with 17G2 suggesting that these proteins indeed function similarly.

Sorting nexin 1 (SNX1) is involved in sorting ligand-activated EGFR to endosomes. SNX1 was identified by a yeast-2-hybrid screen using the kinase domain of human EGFR as bait (Science272:1008-1010). The C-terminal 58 amino acids bind to the EGFR kinase domain. Overexpression of SNX1 resulted in decreased expression of EGFR by enhancing rates of constitutive and ligand-induced degradation. Originally, the only similar sequence reported in GENBANK was that of Mvp1, a yeast protein identified by a genetic screen for modifiers of VPS1 mutants (MCB 15:1671-1678). VPS1 is an 80 kDa GTPase that associates with golgi membrane and is required for the sorting of proteins to the yeast vacuole. MVP1 overexpression suppressed dominant alleles of VPS1. MVP1 is a 59 kDa hydrophilic protein which was also shown to be necessary for protein sorting to yeast vacuoles.

Having illustrated and described the principles of the invention in a preferred embodiment, it should be appreciated to those skilled in the art that the invention can be modified in arrangement and detail without departure from such principles. All modifications coming within the scope of the following claims are claimed.

All publications, patents and patent applications referred to herein are incorporated by reference in their entirety to the same extent as if each individual publication, patent or patent application was specifically and individually indicated to be incorporated by reference in its entirety.

Detailed Figure Legends:

FIG. 1. K17G2-lacZ expression in vitro and in vivo. Overnight X-gal staining showed fusion transcript expression at medium intensity in most undifferentiated K17G2 ES cells (A). The fusion transcript was expressed in the blood island and some of the associated vascular endothelium in attached EB culture (B). Differentiation of clone K17G2 on op9 stromal cells demonstrated lacZ expression in mesodermal colonies (C) and hematopoietic clusters (D). X-gal staining of an e10.5 F₁ embryo demonstrated limited lacZ expression in the embryo (whole mount, E) including expression in the myocardium (F) and the dorsal root ganglia (G). An X-gal stained e12.5 F₁ embryo demonstrated lacZ expression in the endocardium (H) and vascular endothelium and circulating hematopoietic cells (I).

FIG. 2. GC11E10-lacZ expression. Overnight X-gal staining showed fusion transcript expression at medium to high levels in most undifferentiated ES cells (A). In attached EB cultures, lacZ was expressed within blood islands and the associated vascular endothelium (B). Differentiation of clone GC11 E10 on op9 stromal cells demonstrated lacZ expression in mesodermal colonies (C) and a proportion of hematopoietic clusters (D). Overnight whole mount X-gal staining of an e9.5 chimeric embryo and yolk sac demonstrated lacZ expression in the dorsal aorta, heart, liver, and vasculature (E). LacZ expression in the yolk sac was confined to endothelial and hematopoietic cells (F&G). LacZ was expressed by the endocardium and circulating blood cells in the heart (H) and by the intersomitic endothelial cells (I).

FIG. 3. Mena-lacZ (K18E2) expression. Overnight X-gal staining demonstrated high-level lacZ expression in undifferentiated ES cells (A) and in virtually all cells in the attached EB culture including blood islands and their associated vasculature (B). Differentiation of clone K18E2 on op9 stromal cells followed by overnight X-gal staining demonstrated high level lacZ expression in mesodermal colonies (C), whereas most hematopoietic cells did not express lacZ (thick arrows) although low-level expression was observed in some isolated hematopoietic cells (thin arrows, D). Mena-lacZ was expressed at high levels in vivo as demonstrated by strong X-gal staining in less than 90 minutes in an e10.5 F₁ embryo (E). Overnight X-gal staining of an e13.5 F₁ embryo showed strong lacZ expression in all tissues except the liver (F).

TABLE 1
Summary of attached EB primary gene trap screen.

| | | EMBRYOID | | |
|---|---|---|---|---|
| VECTOR | UNDIFFERENTIATED | BODIES | NUMBER (%) | |
| PT1 | BLUE¹ | BLUE | 30 | (4) |
| GT18.geo | | | 159 | (31) |
| PT1 | BLUE | WHITE | 7 | (1) |
| GT18.geo | | | 13 | (3) |
| PT1 | WHITE | BLUE | 61 | (8) |
| GT18.geo | | | 181 | (35) |
| PT1 | WHITE | WHITE | 681 | (87) |
| GT18.geo | | | 156 | (31) |

| | PT1 | GT1.8geo |
|---|---|---|
| Total Number of Neo^{R} Clones | 779 (100) | 509 (100) |
| Total BLUE Clones | 98 (13) | 353 (69) |
| Identifiable Patterns Among β-gal | 32 (33) | 47 (13) |
| positive Clones² | | |

¹“BLUE” indicates detectable β-gal activity.
²Percentage was determined by dividing the number of clones with identifiable patterns of lacZ expression by the total number clones demonstrating β-gal activity.

TABLE 2
Patterns of expression in attached EBs.

| TYPE | PT1-ATG | GT1.8 |
|---|---|---|
| BLOOD ISLAND* | 31% | 40% |
| ENDOTHELIAL | 3% | 4% |
| BLOOD ISLAND AND ENDOTHELIAL* | 3% | 19% |
| STROMA | 3% | 4% |
| MUSCLE | 6% | 0% |
| CONSTITUTIVE | 9% | 19% |
| UNKNOWN CELL TYPE | 45% | 13% |

*70% of clones expressing lacZ in blood islands express lacZ in hematopoietic cells in op9 induction assay.

TABLE 3
Race product analysis.

| | | LacZ Epression Pattern | | |
|---|---|---|---|---|
| Clone | Vector | In Vitro¹ | In Vivo² | Identity |
| K17B1 | PT1-ATG | muscle | muscle, | novel ORF |
| | | | endoderm | |
| K17G2 | PT1-ATG | hematopoietic, | hematopoietic, | human EST |
| | | vascular | vascular, | |
| | | blood island | nervous system, | |
| | | | myocardium | |
| K18E2 | PT1-ATG | constitutive | constitutive | Mena |
| | | | except | |
| | | | hepatocytes | |
| K18F3 | PT1-ATG | muscle | myocardium | novel ORF |
| K20D4 | PT1-ATG | vascular | N.D. | endothelial |
| | | | | EST |
| B2C3 | GT1.8geo | hematopoietic, | N.D. | Karyopherin |
| | | vascular | | β3 |
| B2D2 | GT1.8geo | blood island, | N.D. | embryo EST |
| | | vascular | | |
| GC10A2 | GT1.8geo | hematopoietic, | N.D. | novel ORF |
| | | blood island | | |
| GC10G7 | GT1.8geo | vascular | N.D. | 5′GMP |
| | | | | synthetase |
| GC11C7 | GT1.8geo | hematopoietic | heart, forebrain, | ES cell and |
| | | | otic and optic | placenta |
| | | | vesicles, | ESTs |
| | | | mandibular | |
| GC11E10 | GT1.8geo | hematopoietic, | hematopoietic, | novel ORF |
| | | blood island | vascular | |
| | | vascular | heart | |

¹In vitro analysis was performed by analysis of attached EB cultures and op9 cultures.
²In vivo analysis was performed using diploid or tetraploid aggregation chimeric or F₁ embryos and sacrificing between e9.5 and e14.5.

We claim:

1. A method of identifying a target nucleic acid molecule primarily expressed in selected lineages comprising:
(a) integrating into a site in the genome of a host cell a gene trap vector containing a reporter gene, to form transfected cells;
(b) growing the transfected cells in vitro under conditions whereby the transfected cells differentiate into embryoid bodies attached to a carrier and identifying embryoid bodies expressing the reporter gene in cells of a selected lineage, or
(c) growing the transfected cells in vitro under conditions whereby the transfected cells differentiate into cells of a selected lineage, and identifying cells of the selected lineage expressing the reporter gene;
wherein the target nucleic acid molecule comprises sequences upstream or downstream of the site of integration of the reporter gene in the cells of the selected lineage.

2. A method as claimed in claim l, which further comprises isolating nucleic acid molecules from the transfected cells, or descendents thereof expressing the reporter gene wherein the nucleic acid molecules comprise the reporter gene and a part of the target nucleic acid molecule, or the nucleic acid molecules comprising genomic DNA upstream or downstream of the site of insertion of the gene trap vector.

3. A method as claimed in claim 1, which further comprises forming a chimeric embryo with cells of the selected expressing the reporter gene.

4. A method as claimed in claim 3, wherein the chimeric embryo is allowed to mature to term and mated to provide animal lines or the chimeric embryo can be implanted in a foster recipient females and mated to provide animal lines.

5. A clone expressed primarily in hematopoietic, endothelial, stromal, and/or myocyte lineages designated 17G2, K18F2, K20D4, K18F2, K20D4, B2D2, GC10E10 , GC11C7, and GC11E10.

6. An isolated nucleic acid molecule which comprises:
(i) a nucleic acid sequence encoding a protein having substantial sequence identity preferably at least 75% sequence identity, with the amino acid sequenceof SEQ. ID. NO.2, SEQ. ID. NO 5.,or SEQ. ID. NO.7;
(ii) nucleic acid sequences complementary to (i);
(iii) a degenerate form of a nucleic acid sequence of (i);
(iv) a nucleic acid sequence comprising at least 18 nucleotides and capable of hybridizing to a nucleic acid sequence in (i), (ii), or (iii);
(v) a nucleic acid sequence encoding a truncation, an analog, an allelic or species variation of a protein comprising the amino acid sequence shown SEQ. ID. NO.2, SEQ. ID. NO 5., or SEQ. ID. NO.7; or
(vi) a fragment, or allelic or species variation of (i), (ii) or (iii).

7. A nucleic acid molecule comprising:
(i) a nucleic acid sequence comprising the sequence of SEQ. ID. NO.1, SEQ. ID. NO 3., SEQ. ID. NO. 4, SEQ. ID. NO. 6, SEQ. ID. NO.8, SEQ. ID. NO. 9, or SEQ. ID. NO. 10, wherein T can also be U;
(ii) nucleic acid sequences complementary to (i), sequenceof SEQ. ID. NO.1, SEQ. ID. NO 3., SEQ. ID. NO. 4, SEQ. ID. NO. 6, SEQ. ID. NO. 8, SEQ. ID. NO. 9, or SEQ. ID. NO.10;
(iii) a nucleic acid capable of hybridizing to a nucleic acid of (i) and having at least 18 nucleotides; or
(iv) a nucleic acid molecule differing from any of the nucleic acids of (i) to (iii) in codon sequences due to the degeneracy of the genetic code.

8. An isolated nucleic acid molecule which encodes a 17G2 Protein which comprises:
(i) a nucleic acid sequence encoding a protein having the amino acid sequence of SEQ. ID. NO.1;
(ii) nucleic acid sequences complementary to (i); or
(iii) a nucleic acid capable of hybridizing under stringent conditions to a nucleic acid of (i).

9. A vector comprising a nucleic acid molecule as claimed in claim 7 and the necessary elements for the transcription and translation of the inserted coding sequence.

10. A host cell containing a vector as claimed in claim 9.

11. A method for preparing a protein comprising
(a) transferring a vector as claimed in claim 9 into a host cell;
(b) selecting transformed host cells from untransformed host cells;
(c) culturing a selected transformed host cell under conditions which allow expression of the protein; and
(d) isolating the protein.

12. An isolated protein comprising the amino acid sequence of SEQ. ID. NO.2, SEQ. ID. NO 5., or SEQ. ID. NO. 7

13. Antibodies having specificity against an epitope of a protein as claimed in claim 12.

14. A probe comprising a sequence derived from a nucleic acid molecule as claimed in claim 7.

15. A method for identifying a substance which binds to a protein as claimed in claim 12 comprising reacting the protein with at least one substance which potentially can bind with the protein, under conditions which permit the formation of complexes between the substance and protein and assaying for complexes, for free substance, for non-complexed protein, or for activated protein

16. A method for evaluating a compound for its ability to modulate the biological activity of a protein as claimed in claim 12 which comprises providing a known concentration of the protein, with a substance which binds to the protein and a test compound under conditions which permit the formation of complexes between the substance and protein, and assaying for complexes, for free substance, for non-complexed protein, or for activated protein.

17. A composition comprising one or more of a protein as claimed in claim 12, or a substance or compound identified using a method as claimed in claim 16, and a pharmaceutically acceptable carrier, excipient or diluent.

18. A method for treating or preventing a condition requiring modulation of hematopoiesis, the sensory nervous system, myocardium, or cardiac or neural vasculature comprising administering to a patient in need thereof, a protein as claimed in claim 12 or a composition as claimed in claim 17.


SEQUENCE LISTING (10 sequences)

SEQ ID NO: 1 - DNA, 952 bp, Mus musculus
cggcaccaagcgtctggagccaagagctcggccacggtgagccgcaacctcaatcgtttctccaccttcgtcaagtcgggcggggaggccttcgtgctgggagaggcgtcaggcttcgtgaaggatggggacaagctgtgcgtggtgctgggtccctacggccccgagtggcaggagaacccctaccccttccagtgcaccatcgacgaccccaccaagcagaccaagttcaagggcatgaagagctacatctcttacaagctggtgccccacgcataccccaggtgccccgtgcacaggcgctataagcacttcgattggctgtatgcgcgcctggcggagaaattcccagtcatctcggtgccccatctgcctgagaagcaggccaccgggcgcttcgaagaggacttcatctccaaacgcaggaagggtctgatctggtggatgaaccacatggccagccacccggtgctggcgcagtgcgacgtcttccagcatttcctgacctgccccagcagcactgatgagaaggcctggaaacagggtaagcggaaggctgagaaggatgagatggtgggcgccaacttcttcctcactctgagcaccccacctgctgccgccctggacctgcaggaggtggagagmaagatcgatggcttcaaatgcttcaccaagaagatggacgacagcgcgttgcagctcaaccacaccgccaacgagtttgcgcgcaagcaggtgactggcttcaagaaggagtatcagaaggtgggccagtccttccggggtctcagccaagcctttgagctggatcagcgggccttctccgtgggtctgaatcaggccattgccttcactggagacgcctacgacgccatcggcgaactcttcgctgagcagcccaggcaggacctggacccagtcatggacctgttagcactgtatcgggggcccg

SEQ ID NO: 2 - protein, 317 aa, Mus musculus
  UNSURE (215): Unknown
RHQASGAKSSATVSRNLNRFSTFVKSGGEAFVLGEASGFVKDGDKLCVVLGPYGPEWQENPYPFQCTIDDPTKQTKFKGMKSYISYKLVPHAYPRCPVHRRYKHFDWLYARLAEKFPVISVPHLPEKQATGRFEEDFISKRRKGLIWWMNHMASHPVLAQCDVFQHFLTCPSSTDEKAWKQGKRKAEKDEMVGANFFLTLSTPPAAALDLQEVEXKIDGFKCFTKKMDDSALQLNHTANEFARKQVTGFKKEYQKVGQSFRGLSQAFELDQRAFSVGLNQAIAFTGDAYDAIGELFAEQPRQDLDPVMDLLALYRGP

SEQ ID NO: 3 - DNA, 63 bp, Mus musculus
aatcagagaaggcaatggcttgtgattggtggagggggctgatcatgggaagaggaaccgaaa

SEQ ID NO: 4 - DNA, 435 bp, Mus musculus
aattcggatccaacgcggacgccggtctcatgaatgaaacaatggctacagattctcctcggagacccagtcgttgtactggcggagtcgtggtccgccctcaggccgtcacggagcagtcctacatggagagcgtcgtgacttttctgcaggatgttgtgccacaggttacagtgggtctcccctaacagaagaaaaggagaagatagtctgggtcagatttgagaatgcagatctgaacgacacatcacggaatctagaatttcatgaactgcatagcactggaaatgagcctcctctgctggtgatgatcggctattttgacggaatgcaggtctggggcatccctatcagcggggaagcccaggagctcttctctgtacgacatggtccagtccgagcagctagaatcttgcctgctccacagttgggtgc

SEQ ID NO: 5 - protein, 131 aa, Mus musculus
NNGYRFSSETQSLYWRSRGPPSGRHGAVLHGERRDFSAGCCATGYSGSPLTEEKEKIVWVRFNADLNDTSRNLEFHELHSTGNEPPLLVMIGYFDGMQVWGIPISGEAQELFSVRHGPVRAARILPAPQLG

SEQ ID NO: 6 - DNA, 399 bp, Mus musculus
ctgtcctgacgtcatttcccgtcaaggtactgcttccgggtgtcggcctgctggcgctcgtgtgtgggtgacatcttggcgatcgcttggaagctgccctctttcccctccccgcttcccgcgttgtccgctgtgcctgtctctggggtcctctcccggcctctaccccgggtccgctcccagcgttgccgcctccatcgtgaggtagttgaaatgtaaaagtcggggcctgaagagataactcagcaggaactatgaatgggagggctgattttcgagaaccgaatgcacaagtgtcaagacctattcccgacataggagcgttatattccgacagaggaggagtggagactctttgcagagtgcatgaagagtgcttcttggctagagttccagtct

SEQ ID NO: 7 - protein, 55 aa, Mus musculus
RDNSAGTMNGRADFREPNAQVSRPIPDIGALYSDRGGVETLCRVHEECFLARVPV

SEQ ID NO: 8 - DNA, 334 bp, Mus musculus
cttgggccagacgccaacgtcaccagccaggtactcacccatttctaaagccgtgctcggagatgacgagatcactagggaacctagaaaagttgttcttcatcgtggctcaacaggacttggttttaacattgtgggaggtgaagatggagaagggatttttatctccttcayccttgctggcggacctgctgatctaagtggagagctcagaaaaggagatcgcatcatatcggtgaacagtgttgacctcagagctgcaagtcacgaacaagcagaagctgcactaaagaacgcaggccaagccgtcaccatcgttgcacaatatcgaccc

SEQ ID NO: 9 - DNA, 53 bp, Mus musculus
aaatcgaacaggagctgacggctgccaagaagcacggcaccaaaataagcgcg

SEQ ID NO: 10 - DNA, 105 bp, Mus musculus
ggggcgtcccagaaamagctggcactctgtattccacagggtcaccgtgmagcctgccctccgcggagtcccggagccaagaattcatgggaagaggaaccgaaa